(19) United States
(12) Patent Application Publication
(10) Pub. No.: US 2016/0249851 A1
(43) Pub. Date: Sep. 1, 2016

(54) Digitally Coached Spirometry System and Method

(71) Applicant: 12th Man Technologies, Inc., Garden Grove, CA (US)

(72) Inventor: Alex Stenzler, Long Beach, CA (US)

(21) Appl. No.: 15/053,675

(22) Filed: Feb. 25, 2016

Related U.S. Application Data
(60) Provisional application No. 62/126,022, filed on Feb. 27, 2015.

(51) Int. Cl. A61B 5/00; A61B 5/087

(52) U.S. Cl. CPC A61B 5/486; A61B 5/087; A61B 5/742; A61B 5/741

(57) ABSTRACT

A spirometry coaching system and method is described. The system includes a spirometer comprising at least one detection element, a patient interactive display, and a control unit communicatively connected to the detection element and the patient interactive display, wherein the control unit directs an audio and visual presentation of at least one test instruction on the patient interactive display, and wherein the control unit directs an audio and visual presentation of at least one test result on the patient interactive display based on a measurement received from the at least one detection element.

### CROSS-REFERENCE TO RELATED APPLICATIONS

This application claims benefit and is entitled to priority under 35 U.S.C. §119(e) to U.S. Provisional Patent Application No. 62/126,022, filed Feb. 27, 2015, which application is hereby incorporated herein by reference in its entirety.

### BACKGROUND OF THE INVENTION

Spirometry is a common and well established pulmonary function test for evaluating patients with conditions such as asthma, cystic fibrosis, pulmonary fibrosis and COPD. The testing assesses function of the patient's lungs by measuring the volume and flow of air that a patient is able to inhale and exhale.

Typically, spirometry is performed at a physician's office or a pulmonary function laboratory in the presence of a medical professional such as a doctor, nurse or medical technician. The testing is performed using a spirometer, which generally requires the patient to take a very deep breath and exhale as hard as possible for as long as possible into the spirometer's mouthpiece. The testing is performed according to standards that are generally adopted in the medical field (see for example, Miller et al., “Standardisation of spirometry”, EUR Respir J 2005; 26: 319-338; herein incorporated by reference). The accuracy of the testing is highly dependent on the cooperation of the patient, the ability of the patient to understand and follow instructions, and the ability of the patient to execute the breathing maneuvers at a high level. If the breathing maneuvers are performed poorly, there is an increased risk that the spirometry results will be misinterpreted, potentially leading to an erroneous result.

One of the limiting factors in the success of performing lung function spirometry measurements in a patient's home is that there is not a technologist there to coach the patient as there would be if the test was performed in a physician's office or pulmonary function laboratory. For instance, in a home setting, patients do not necessarily know when to breathe quietly, when to take a deep breath, or when and how long to forcefully exhale. Further, home spirometry measurement devices lack the verbal encouragement and visual aids that would enable a test to produce a consistent and reliable measurement for the patient. Additionally, subjects will routinely perform multiple measurements, which is tiring and time consuming. Accordingly, spirometry self-testing at home is known to be inadequate (see for example, Pelkonen et al., “Reproducibility of Home Spirometry in Children With Newly Diagnosed Asthma,” Pediatric Pulmonology, 2000, 29:34-38; Wensley and Silverman, “The quality of home spirometry in school children with asthma,” Thorax, 2001, 56:183-185; Thompson et al., “Evaluation of Daily Home Spirometry for School Children with Asthma: New Insights,” Pediatric Pulmonology, 2006, 41:819-828; all of which are herein incorporated by reference).

Software packages have been created to help and encourage patient performance during spirometry testing. For instance, certain systems have utilized a visual stimulus such as simulating the blowing-out of candles or a game involving a caterpillar crawling to an apple for encouraging patient performance (see for example, Vilozini et al., “An Interactive Computer-Animated System (SpiroGame) Facilitates Spirometry in Preschool Children”, Am J Respir Crit Care Med Vol 164. pp 2200-2205, 2001). However, these images and visual feedback are typically accompanied by a live technologist providing coaching to the patient. In other examples, U.S. Pat. No. 6,126,613 to Edwards et al. generally discloses a voice system with prerecorded instructions to walk the user through the use of the spirometer. U.S. Pat. No. 7,591,789 to Bryant generally discloses using audio for providing the user with various instructions, guidance and measurement feedback. However, both Edwards et al. and Bryant fail to provide a system and method for real-time, comprehensive, interactive and patient-specific instruction and feedback prior to, during and after the actual breathing maneuvers for achieving accurate and reliable spirometry testing results, sufficient for effectively replacing a real-life medical professional.

Thus, what is needed is a spirometry system and method that effectively coaches the patient during the maneuver using immediate and real time feedback, so that accurate and reliable test results can be obtained in a home setting and outside the presence of a medical professional. Further, what is needed is a consistent and reliable system to reduce the number of measurements taken, thereby minimizing the time and fatigue experienced by patients with chronic lung disease who perform the spirometry testing.

### SUMMARY OF THE INVENTION

A spirometry coaching system and method is described. The system includes a spirometer comprising at least one detection element, a patient interactive display, and a control unit communicatively connected to the detection element and the patient interactive display, wherein the control unit directs an audio and visual presentation of at least one test instruction on the patient interactive display, and wherein the control unit directs an audio and visual presentation of at least one test result on the patient interactive display based on a measurement received from the at least one detection element. In one embodiment, the control unit further directs an audio and visual presentation of at least one pre-test instruction on the patient display. In another embodiment, the control unit further directs an audio and visual presentation of at least one post-test information item on the patient display. In another embodiment, the post-test information item is based on the at least one test result. In another embodiment, the detection element is a transducer. In another embodiment, the at least one test result corresponds to flow rate. In another embodiment, the flow rate is presented over a 6 s time period. In another embodiment, the time period corresponding to the first second of time is expanded. In another embodiment, the 120 ms timepoint is identified.

A method for coaching a spirometry test is also described. The method includes the steps of presenting at least one audio and visual test instruction on a patient interactive display, measuring at least one parameter of a breathing maneuver performed by a patient using a spirometer, calculating a value based on the measured parameter, and presenting at least one audio and visual test result on the patient interactive display based on the calculated value. In one embodiment, the method further includes the step of presenting at least one audio and visual pre-test instruction on the patient interactive display. In another embodiment, the method further includes the step of presenting at least one audio and visual post-test information item on the patient interactive display. In another embodiment, the post-test information item is based on the test result. In another embodiment, the at least one test result corresponds to flow rate. In another embodiment, the flow rate is presented over a 6 s time period. In another embodiment, the time period corresponding to the first second of time is expanded. In another embodiment, the 120 ms timepoint is identified. In another embodiment, the test result is a time-to-peak flow rate. In another embodiment, the post-test information item is an instruction that the maximum flow rate was reached too late. In another embodiment, the post-test information item is an instruction that the maximum flow rate was not reached.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of an exemplary spirometry system according to an aspect of the invention.

FIG. 2 is an exemplary introduction program flow according to an aspect of the invention.

FIG. 3 is an exemplary pre-test instruction program flow according to an aspect of the invention.

FIGS. 4A-4C show exemplary test instruction program flows according to aspects of the invention. FIGS. 4A and 4B are test instructions and protocols. FIG. 4C is graphical user interface for a test screen.

FIGS. 5A and 5B are exemplary post-test information program flows according to aspects of the invention.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein is system and method for coached spirometry.

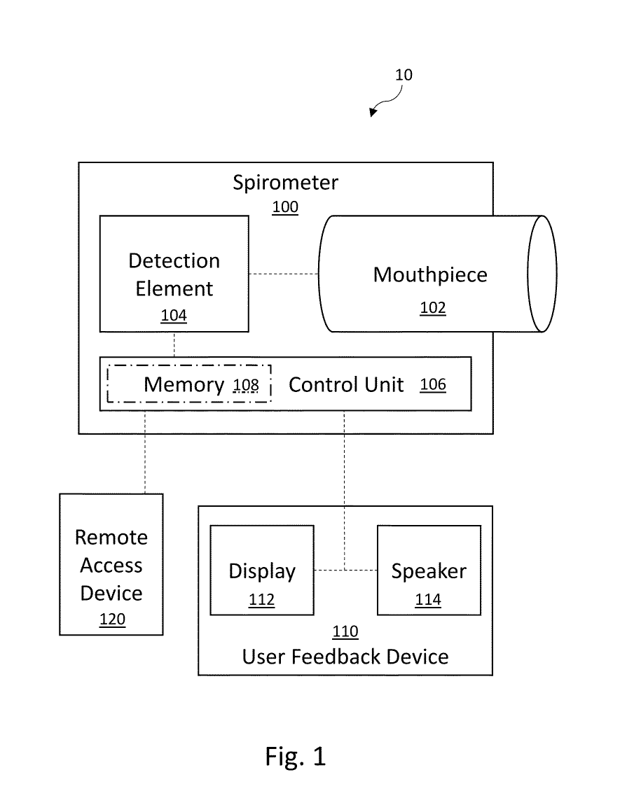

With reference to FIG. 1, system 10 may include a spirometer 100 that has a mouthpiece 102 opening for the patient to perform breathing maneuvers into. Generally, the patient takes a very deep breath and exhales as hard as possible for as long as possible into the mouthpiece 102. At least one detection element 104 is used to detect airflow rates from the patient. Different types of detection elements can be used to detect airflow. For instance, a transducer, such as an ultrasonic transducer can be used to generate an electrical measurement signal that measures the speed of airflow or a pressure difference in a channel. Other types of detection elements known in the art for spirometry, such as windmill-type mechanisms or a pneumotachometer can also be used to detect airflow characteristics and generate electrical signals indicative of the measurement. The detection element 104 is connected to a control unit 106, which processes a signal from the detection element 104 for calculating values such as volume and airflow rates. The control unit 106 can also include a memory 108 for storing software that executes the methods described herein. The memory 108 also stores measured values and calculated values so that the software can recall information from breathing maneuvers and utilize this information for making real-time or post-testing decisions on how to instruct the patient. In certain embodiments, the control unit 106 is integrated into the handheld spirometer 100. In other embodiments, the control unit 106 is part of a separate computing device, such as a laptop, tablet or smart phone, or it may alternatively be housed in user feedback device 110. It should be appreciated that the control unit 106 may be positioned in any type of computing device as would be understood by those skilled in the art, provided such computing device is connected to one or more detection elements 104 of spirometer 100 via a communications network. As contemplated herein, any spirometer, user feedback device or other computing device may generally include at least one processor, standard input and output devices, as well as all hardware and software typically found on computing devices for storing data and running programs, and for sending and receiving data over a network. Accordingly, control unit 106 may freely communicate with a remote access device 120. The remote access device 120 may be any type of computing device described herein, and can be used by an off-site medical professional for setting certain parameters, thresholds and ranges disclosed herein. The control unit 106 can also send test results to the remote access device 120 so that results can be monitored and viewed by the off-site medical professional, even though the patient is able to complete the testing by themselves, entirely within the home care setting. The control unit 106 also communicates with a user feedback device 110 having at least a display 112 and speaker 114 to present audio or visual instructions, test results, or other types of data or information disclosed herein. As contemplated herein, user feedback device 110 may also be any sort of computing device described herein, including desktop or moble devices, laptops, tablets, wireless digital/cellular phones, smart phones, televisions or other thin client devices as would be understood by those skilled in the art. In certain embodiments, a component of the user feedback device, such as the speaker 114, may be integrated into the spirometer 100. System preferences and other types of information can be sent to the control unit 106 through an input interface integrated with or connected to the user feedback device 110 such as a keyboard, touchscreen or voice command module.

Without limitation, other additional computing devices may be used with the system, including desktop or moble devices, laptops, tablets, wireless digital/cellular phones, televisions or other thin client devices as would be understood by those skilled in the art.

Further, the system 10 has associated therewith a software platform that may operate as a local or remote executable software platform. For example, the computer operable component(s) of the system may reside entirely on a single computing device, or may reside on any number of devices within the system. Similar to control unit 106, remote access device 120 and user feedback device 110, any computing devices contemplated herein may generally include at least one processor, standard input and output devices, as well as all hardware and software typically found on computing devices for storing data and running programs, and for sending and receiving data over a network. Any computing device forming part of the system 10 may also be connected directly or via a network to remote databases, such as for additional storage backup, and to allow for the communication of files, email, software, and any other data format between two or more computing devices. There are no limitations to the number, type or connectivity of the databases utilized by the system of the present invention.

The system 10 may include a communications network as would be understood by those having ordinary skill in the art, such as, for example, an open, wide area network (e.g., the internet), an electronic network, an optical network, a wired or wireless network, a physically secure network or virtual private network, connection and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system 10.

Further, the communications network may use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access.

As mentioned previously, the system may include an application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system may provide software accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal or SaaS that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet or from a limited or restricted database. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed folder files, or other layering techniques understood by those skilled in the art or through a novel natural language interface as described herein. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a user of the particular results. Further embodiments of such mechanisms are described elsewhere herein or may standard systems understood by those skilled in the art.

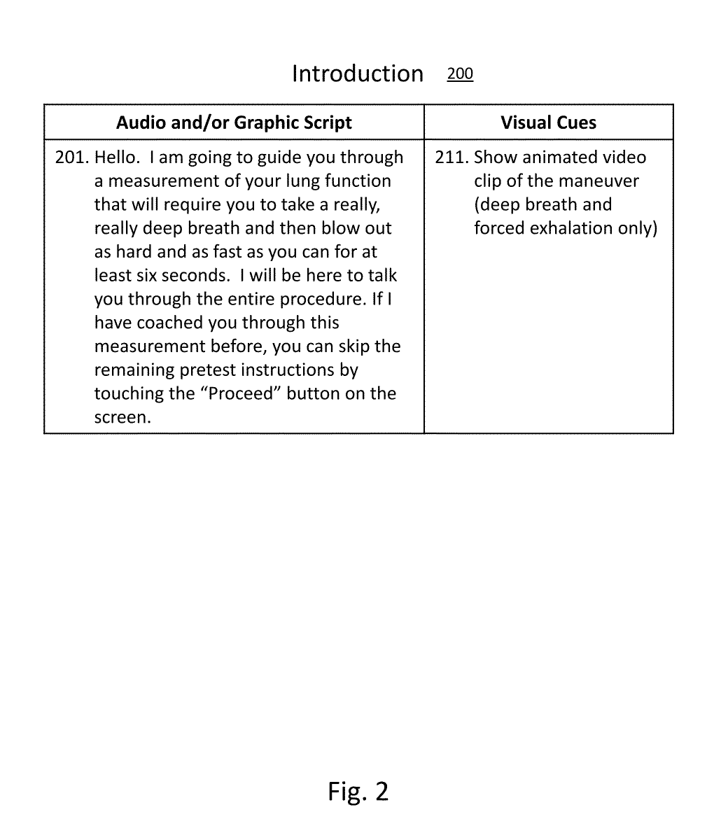

Accordingly, the system may include a gamification script to guide the user through the spirometry testing. Using the aforementioned computing device platform and network, the script can be communicated to the patient using one or both of an audio communication medium, such as the speaker 114, and a GUI, such as the display 112. Visual cues such as graphical animations may accompany portions of the script. A non-limiting, exemplary embodiment of a script and program flow with corresponding visual cues for obtaining accurate and reliable spirometry test results in a home setting is disclosed in FIGS. 2-5B.

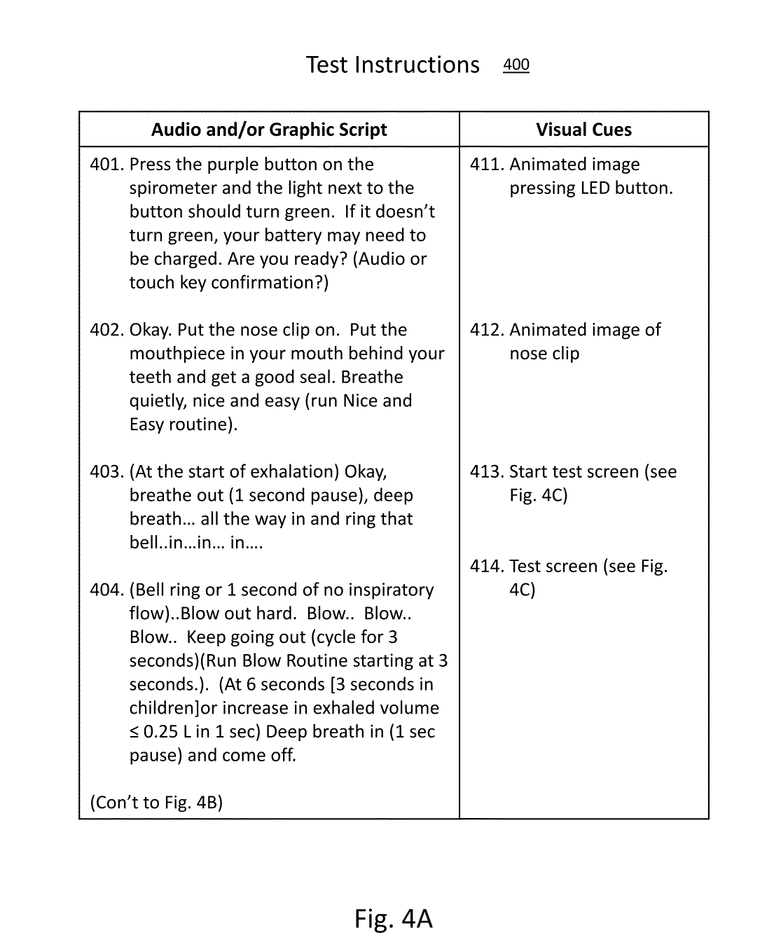

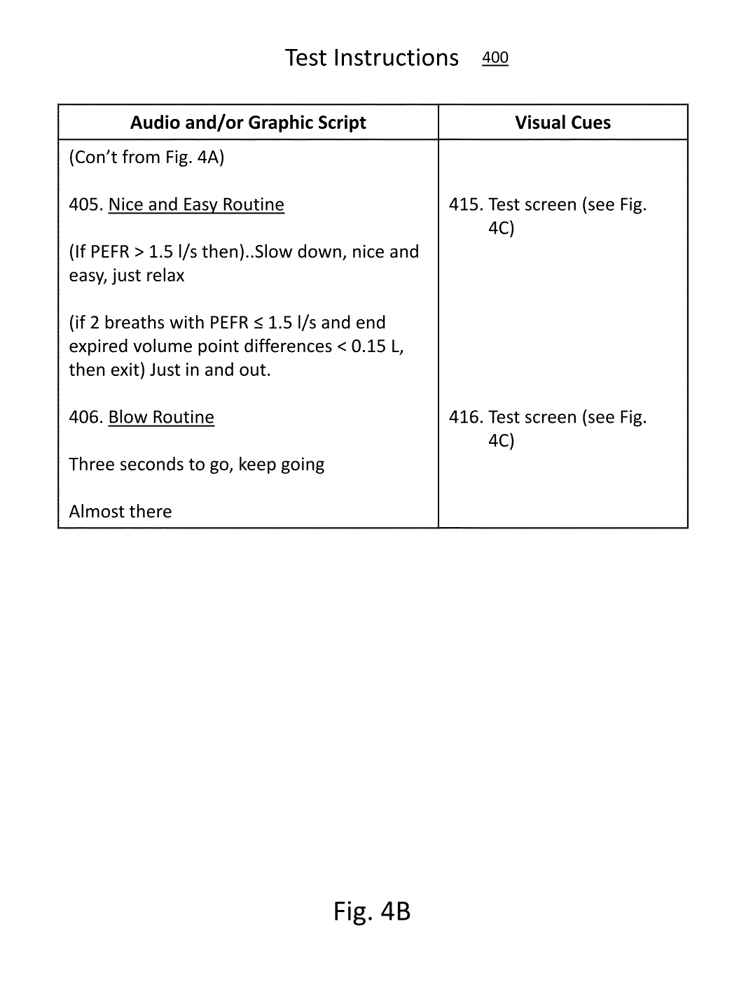

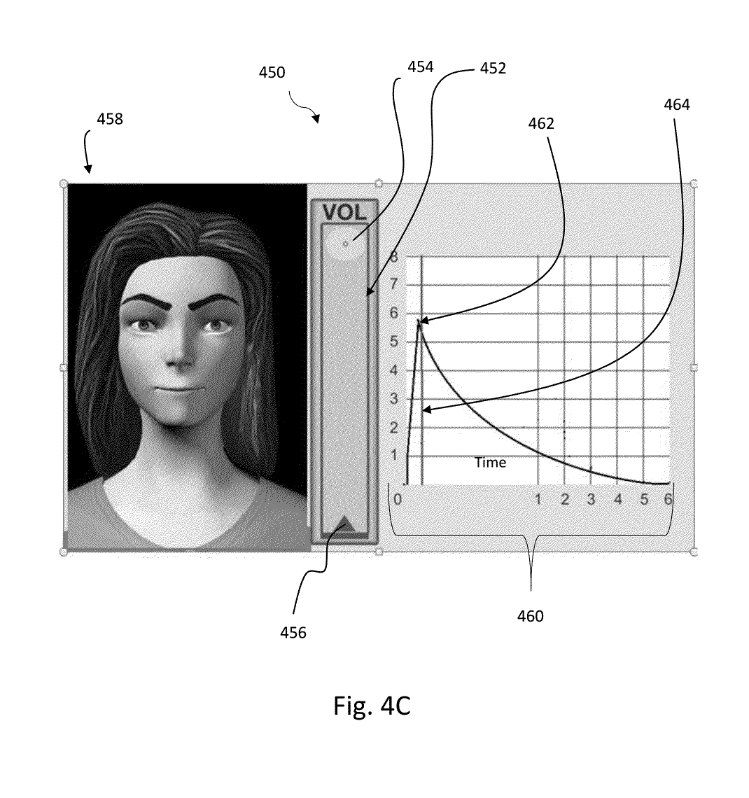

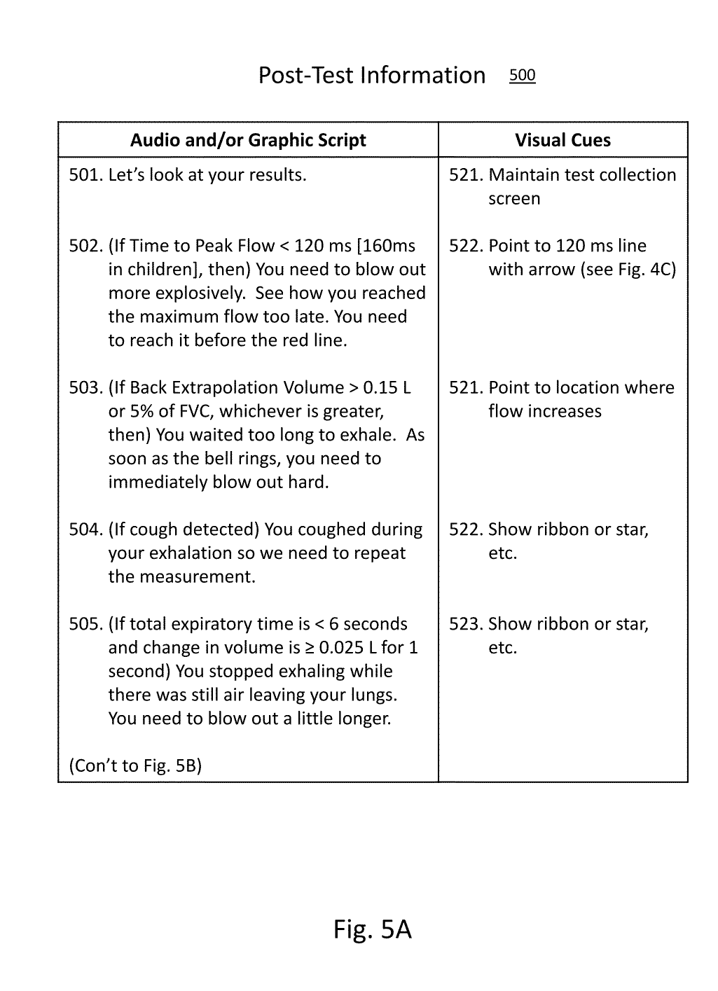

For example, and with reference to FIG. 2, an introduction set 200 that includes at least a script 201 and a set of visual cues 211 is executed to familiarize the patient with the software, equipment and spirometry test. Without limitation, the script 201 generally introduces the patient to the purpose of the test, the general requirements of the breathing maneuver, the role of the software as a “digital coach”, and options that the patient will have to skipping one or more pre-test instructions. In one embodiment, the script may read: “Hello. I am going to guide you through a measurement of your lung function that will require you to take a really, really deep breath and then blow out as hard and as fast as you can for at least six seconds. I will be here to talk you through the entire procedure. If I have coached you through this measurement before, you can skip the remaining pretest instructions by touching the “Proceed” button on the screen. The corresponding visual cue 211 is an animation of the breathing maneuver, focused on teaching the patient about the deep inhale and forced exhale breathing techniques. As explained above, the software can be set to use one or both of audio and graphical mediums for relaying the script to the patient. In preferred embodiments, the patient will hear a voice accompanied by a facial depiction of their virtual “digital coach” (see for instance FIG. 4C, 458).

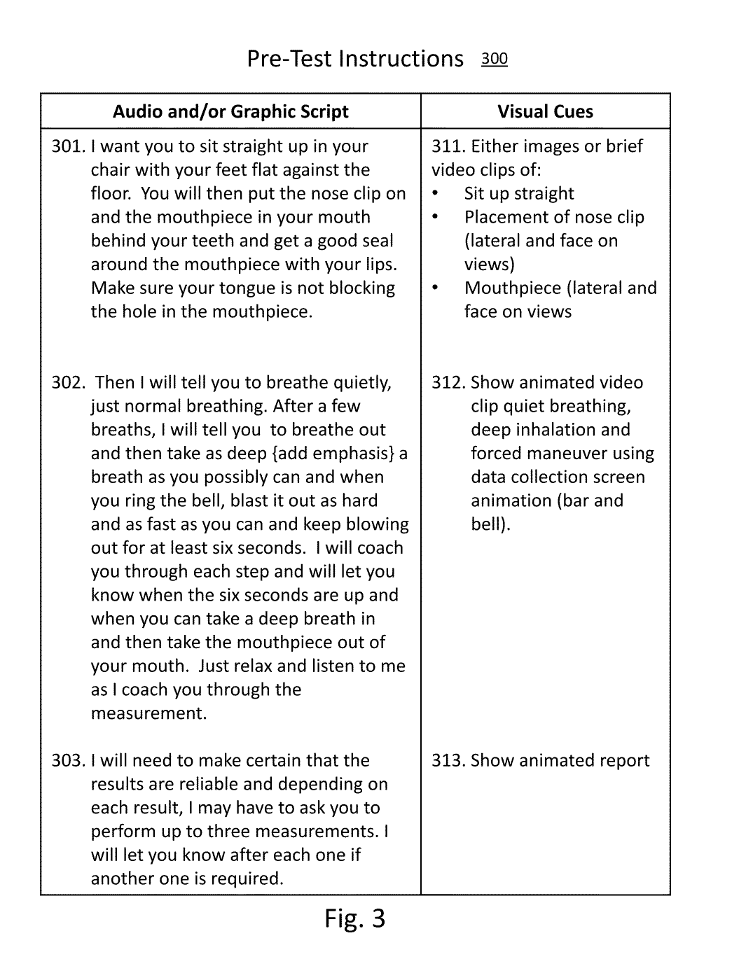

With reference to FIG. 3, optional pre-test instructions 300 may then be provided. The pre-test instructions may include a script 301 instructing the user as to posture, use of spirometry system accessories and technique for interfacing the spirometer mouthpiece with the mouth. In one embodiment, the script may read: “I want you to sit straight up in your chair with your feet flat against the floor. You will then put the nose clip on and the mouthpiece in your mouth behind your teeth and get a good seal around the mouthpiece with your lips. Make sure your tongue is not blocking the hole in the mouthpiece.” The corresponding visual cue 311 shows an animation of a model patient with certain anatomical details that models proper posture, placement of accessories such as the nose clip, and interface with the mouthpiece. Views of visual cues and magnification levels may change automatically, and alternatively views that the patient would prefer or like to repeat for a better understanding of proper technique can be selectable by using an input device such as a touchscreen. The patient is then instructed as to the specifics of a breathing maneuver 302. In one embodiment, the script may read: “Then I will tell you to breathe quietly, just normal breathing. After a few breaths, I will tell you to breathe out and then take as deep {add emphasis} a breath as you possibly can and when you ring the bell, blast it out as hard and as fast as you can and keep blowing out for at least six seconds. I will coach you through each step and will let you know when the six seconds are up and when you can take a deep breath in and then take the mouthpiece out of your mouth. Just relax and listen to me as I coach you through the measurement.” The corresponding visual cue 312 shows a virtual simulation of the various stages of the breathing maneuver, including quiet breathing, deep inhalation and a forced exhalation maneuver. The patient can view the simulated breathing maneuver, and learn about how the system will determine whether or not the results are reliable, and why the system may prompt the user to perform one or more additional breathing maneuvers 303. This helps from discouraging the patient in the event they are later asked to repeat a breathing maneuver. In one embodiment, the additional breathing maneuver 303 script may read: “I will need to make certain that the results are reliable and depending on each result, I may have to ask you to perform up to three measurements. I will let you know after each one if another one is required.” An animated report will pop-up on the display as a visual cue 313, so that the user has a better understanding of how they can interpret their performance results during the testing phase. The animated report will explain how the breathing meters operate and what the graphs show, so that the patient becomes generally familiar with interpreting their performance and test results.

After any pre-test instructions 300 and protocols are completed, the patient and system move ahead to the testing phase and the test instructions 400, as shown in FIGS. 4A-4C. In certain embodiments, much of what the patient is prompted to do during the testing set may track the simulation that they just viewed and the instructions that they just heard previously in the pre-test instructions. Accordingly, the system will first prompt the user to check battery function and confirm that they are ready to begin the test 401. In one embodiment, the script may read: “Press the purple button on the spirometer and the light next to the button should turn green. If it doesn't turn green, your battery may need to be charged. Are you ready? (Audio or touch key confirmation?)”. A corresponding visual cue shows the user where the button is located 411. The user is then instructed to attach system accessories, such as the nose clip 402. In one embodiment, the script may read: “Okay. Put the nose clip on. Put the mouthpiece in your mouth behind your teeth and get a good seal. Breathe quietly, nice and easy (run Nice and Easy routine).” An animated image shows how accessories should be properly placed on the patient 412. Next, the user will be prompted to take steps leading up to their deep inhalation.

The user is then instructed to begin the “nice and easy” breathing routine 402. The “nice and easy” breathing routine is embedded in the software and displayed via GUI 450, as shown in FIG. 4C. The system software may use values extracted in real-time from the breathing measurements to determine the next script for the patient. For example, if quiet breathing peak expiratory flow (PEFR)>1.5 Vs then the script may read, “ . . . Slow down, nice and easy, just relax”. If 2 breaths are executed with PEFR 1.5 Vs and end expired volume point differences<0.15 L, then the script may read, “Just in and out.” At this stage, the patient may be prompted just before an inhalation to take-in a deep exhale 403, while the visual cue changes over to the test screen 413. In one embodiment, the script may read: “Okay, breathe out (1 second pause), deep breath . . . all the way in and ring that bell . . . in . . . in. . . in . . . .”

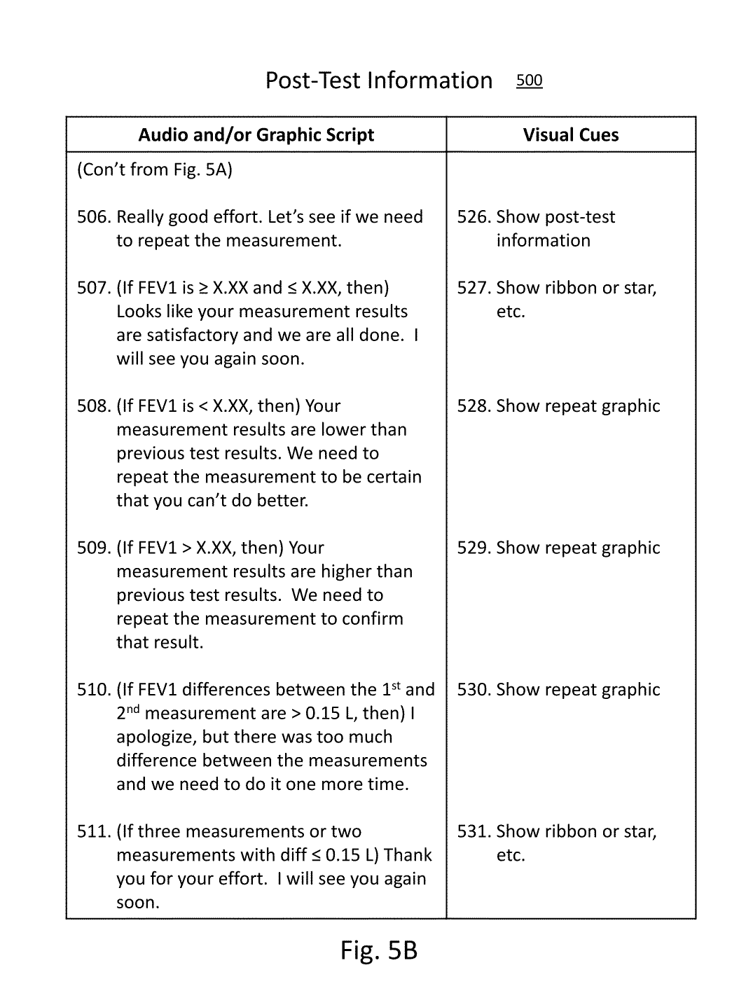

The bell refers to a graphic on the test screen GUI 450, which is shown in FIG. 4C. At various stages of the process, the test screen 450 can include an image of the “digital coach” 458, a bell meter 452, and at least one graph 460 presenting the results of the patient's performance according to at least one metric. In one embodiment, the bell meter 452 may include a float 456 that moves up as measured inhaled volume from the patient increases. When a threshold volume is reached, the bell will ring, signaled to the patient as an audio and/or visual indication. The threshold volume to ring the bell can be set by a medical professional via the remote access device 120 (see FIG. 1), or it may be a preset value, or determined from previously collected data. With reference back to FIG. 4A, once the bell rings, or after one second of zero flow, the patient is prompted to begin blowing out as hard as they can into the mouthpiece 404. This encouragement script can cycle for about 3 seconds while the test screen remains up 414. The script then jumps to the embedded blow routine 406, starting at 3 seconds. The blow routine 406 encourages the patient to make a continuous blow into the spirometer. In one embodiment, the blow routine script may read: “Three seconds to go, keep going; Almost there.” The test screen remains up for both the nice and easy routine 415 and the blow routine 416. At 6 seconds (or 3 seconds for children), or in the event that there is an increase in exhaled volume 0.25 L in 1 sec., the patient is instructed to take a deep breath in and come off the mouthpiece 404. At this point, and with reference now to FIGS. 5A and 5B, the patient is done with the breathing maneuver, and can optionally review their performance by looking at their results 501 by reviewing post-test information 500. The post-test information 500 scripts are dependent on the measurements and calculations determined by the control unit of the system. For example, if the Time to Peak Flow<120 ms [160 ms in children], then the script may read: “You need to blow out more explosively. See how you reached the maximum flow too late. You need to reach it before the red line.” 502. The corresponding visual cue 522 will point to the 120 ms line on graph 460 on the testing screen 450 with an arrow (see FIG. 4C and line 464), so that the patient can see using a visual cue how close they were to maxing-out before 120 ms. If the Back Extrapolation Volume>0.15 L or 5% of FVC, whichever is greater, then the script may read: “You waited too long to exhale. As soon as the bell rings, you need to immediately blow out hard.” 503. The corresponding visual cue 523 will point to the location on the testing screen 450 graph 460 where the flow increases (see FIG. 4C and point 462). A similar visual cue 524, 525, will occur if a cough is detected 504, or if total expiratory time is <6 seconds and change in volume is 0.025 L for 1 second 505. In the latter case 525, a script may read: “You stopped exhaling while there was still air leaving your lungs. You need to blow out a little longer.”

In the embodiment shown in FIG. 4C, graph 460 may depict flow verses time over 6 seconds, and may further be expanded along the time axis between time points 0 and 1 second. This expansion across the first second of time uniquely provides suitable resolution to visualize the measured performance at the 120 ms timepoint, or the 160 ms timepoint for children under 10 years of age. This graphical expansion between the 0 and 1 second timepoints is of particular benefit when patients are self-testing so there is adequate visual resolution feedback for how fast they are reaching their maximal exhaled flow rate. And for example, when this time axis is expanded in the 0-1 second timeframe, the system may visually present to the patient the measurements as outlined above during the post-test information set, there is adequate resolution for the patient to recognize if they have exhaled too slowly.

When the test results are complete and ready for evaluation, the user will be congratulated on their effort, and the control unit will determine whether or not a repeat measurement is required 506. Post-test information will remain on display 526. If the results are satisfactory, the visual cue will change to a congratulatory graphic, such as a show ribbon, a star or another similar type of graphic 527, 531. End of testing may be prompted if, for instance, if FEV1 is within the statistically acceptable range determined from the mean and standard deviation calculated from the previous measurements. The range of acceptable FEV1 to end testing is calculated between Mean FEV1−1.68×SD and Mean FEV1+2.45×SD, 507; or if three measurements or two measurements have a difference of 0.15 L, 511. Ranges can be customized based on patient characteristics, the opinion of the medical professional working with the patient, and past performance values recorded by the control unit. In this case, if satisfied, the script will let the patient know that their results are satisfactory 507, 511. On the contrary, a graphic indicating that the procedure needs to be repeated will show as the visual cue in at least three situations. First, if FEV1 is <Mean FEV1−1.68×SD, then a repeat procedure visual cue will display 528, and the script may read: “Your measurement results are lower than previous test results. We need to repeat the measurement to be certain that you can't do better.” 508. Second, if FEV1>Mean FEV1+2.45×SD, then a repeat procedure visual cue will display 529, and the script may read: “Your measurement results are higher than previous test results. We need to repeat the measurement to confirm that result.” 509. And third, if FEV1 differences between the 1^{st} and 2^{nd} measurement are >0.15 L, then a repeat procedure visual cue will display 530, and the script may read: “I apologize, but there was too much difference between the measurements and we need to do it one more time.” 510. In these cases where a repeat measurement is required, the digital coach and testing screen provide a number of visual and audio insights for the patient to understand how to improve their performance so that a minimal number of repeated breathing maneuvers are required for obtaining accurate and reliable testing results.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A spirometry coaching system comprising:
a spirometer comprising at least one detection element;
a patient interactive display; and
a control unit communicatively connected to the detection element and the patient interactive display,
wherein the control unit directs an audio and visual presentation of at least one test instruction on the patient interactive display, and
wherein the control unit directs an audio and visual presentation of at least one test result on the patient interactive display based on a measurement received from the at least one detection element.

2. The system of claim 1, wherein the control unit further directs an audio and visual presentation of at least one pre-test instruction on the patient display.

3. The system of claim 1, wherein the control unit further directs an audio and visual presentation of at least one post-test information item on the patient display.

4. The system of claim 3, wherein the post-test information item is based on the at least one test result.

5. The system of claim 1, wherein the detection element is a transducer.

6. The system of claim 1, wherein the at least one test result corresponds to flow rate.

7. The system of claim 6, wherein the flow rate is presented over a 6 s time period.

8. The system of claim 7, wherein the time period corresponding to the first second of time is expanded.

9. The system of claim 8, wherein the 120 ms timepoint is identified.

10. A method for coaching a spirometry test, comprising:
presenting at least one audio and visual test instruction on a patient interactive display;
measuring at least one parameter of a breathing maneuver performed by a patient using a spirometer;
calculating a value based on the measured parameter;
presenting at least one audio and visual test result on the patient interactive display based on the calculated value.

11. The method of claim 10, further comprising presenting at least one audio and visual pre-test instruction on the patient interactive display.

12. The method of claim 10, further comprising presenting at least one audio and visual post-test information item on the patient interactive display.

13. The method of claim 12, wherein the post-test information item is based on the test result.

14. The method of claim 13, wherein the at least one test result corresponds to flow rate.

15. The method of claim 14, wherein the flow rate is presented over a 6 s time period.

16. The method of claim 15, wherein the time period corresponding to the first second of time is expanded.

17. The method of claim 16, wherein the 120 ms timepoint is identified.

18. The method of claim 17, wherein the test result is a time-to-peak flow rate.

19. The method of claim 18, wherein the post-test information item is an instruction that the maximum flow rate was reached too late.

20. The method of claim 18, wherein the post-test information item is an instruction that the maximum flow rate was not reached.
